(19) United States
(12) Patent Application Publication
(10) Pub. No.: US 2023/0039728 A1
(43) Pub. Date: Feb. 9, 2023

(54) HEARING ASSISTANCE DEVICE MODEL PREDICTION

(71) Applicant: Starkey Laboratories, Inc., Eden Prairie, MN (US)

(72) Inventors: Olabanji Yussuf Shonibare, Eden Prairie, MN (US); Jingjing Xu, Eden Prairie, MN (US); Tao Zhang, Eden Prairie, MN (US)

(21) Appl. No.: 17/757,685

(22) PCT Filed: Dec. 31, 2020
(86) PCT No.: PCT/US2020/067732

§ 371 Date: Jun. 17, 2022

Related U.S. Application Data
(60) Provisional application No. 62/955,614, filed on Dec. 31, 2019.

(51) Int. Cl. G16H 20/40; G06N 7/00

(52) U.S. Cl. CPC G16H 20/40; G06N 7/005

(57) ABSTRACT

Systems and methods may be used to predict an applicable a hearing assistance device shell or model. For example, a method may include obtaining patient information, determining, using a machine learning trained model, a correlation between an input vector and each of a plurality of feature vectors corresponding to a plurality of hearing assistance device models, and ranking the plurality of hearing assistance device models based on respective correlations to the input vector. Information corresponding to a highest ranked hearing assistance device model may be output.

### CLAIM OF PRIORITY

This application claims the benefit of priority to U.S. Provisional Application No. 62/955,614, filed Dec. 31, 2019, titled “HEARING ASSISTANCE DEVICE SHELL PREDICTION”, which is hereby incorporated herein by reference in its entirety.

### BACKGROUND

Hearing devices provide sound for the wearer. Examples of hearing devices include headsets, hearing assistance devices, speakers, cochlear implants, bone conduction devices, and personal listening devices. Hearing assistance devices provide amplification to compensate for hearing loss by transmitting amplified sounds to their ear canals. In various examples, a hearing assistance devices is worn in or around a patient's ear. Hearing assistance devices have a shell that protects interior components and is shaped to be comfortable for a user.

### BRIEF DESCRIPTION OF THE DRAWINGS

FIG. 1 illustrates examples of hearing assistance devices (e.g., standard or custom) according to an example.

FIGS. 2-3 illustrate a machine learning process for use in predicting a hearing assistance device shell for a patient according to an example.

FIG. 4 illustrates a hearing assistance device dispensing process using a prediction model according to an example.

FIG. 5 illustrates a system for predicting hearing assistance device shell for a patient according to an example.

FIG. 6 illustrates prediction models for clinic and production or manufacturing of hearing assistance devices according to an example.

FIG. 7 illustrates a flowchart showing a technique for predicting a hearing assistance device shell for a patient according to an example.

FIG. 8 illustrates a flowchart showing a technique for training a model to predict a hearing assistance device shell for a patient according to an example.

FIG. 9 illustrates generally an example of a block diagram of a machine upon which any one or more of the techniques discussed herein may be performed according to an example.

### DETAILED DESCRIPTION

Comfort, fit, and quality of sound are important factors in whether a user keeps or returns a hearing assistance device. It may be difficult for a user testing a hearing assistance device in a store to determine whether the hearing assistance device is a good fit (e.g., movement may cause any issue), is comfortable, or has good quality of sound (e.g., due to limited environmental sound). Later identification of issues in fit, comfort, sound quality, or other issues with a hearing assistance device may drive a user to return the hearing assistance device. When hearing assistance device users are unsatisfied with their newly purchased hearing assistance device, they do not wear the devices regularly. Many users even return their hearing aids within trial periods. Returned hearing assistance devices are inconvenient for users, costly for dispensers and manufacturers, and may cause delays.

In clinical practice, hearing assistance device selection is typically based on the past experience of a clinician. However, to make a patient a successful hearing assistance device user, many factors may be helpful other than experience, such as audiological test results, demographic, personality, auditory lifestyle, socioeconomic status, some of which may not be available to a clinician. The systems and methods described herein may assist a clinician in making a data- or information-driven decision on hearing assistance device selection, for example based on sonic or all identified factors.

Systems and methods described herein may be used to predict an applicable hearing assistance device shell for a patient. The prediction may be made by a generated trained model, for example using machine learning techniques. The model may be trained using past hearing assistance device data, such as including the factors listed above, information about whether the hearing assistance device was returned, or the like.

A prediction may be based on available semi-customized hearing assistance device shells (e.g., such that most users (e.g., 90-95%) are able to use one of without discomfort). The prediction may be specific to a type of hearing assistance device, for example in-the-ear (ITE), in-the-canal (ITC), completely-in-the-canal (CIC), invisible-in-the-canal (IIC), or the like.

The systems and methods described herein address the technical problem of determining the chances that a hearing assistance device may be returned based on patient data. The solutions presented herein include using past data to train a model using machine learning to predict likelihood of returns for future users. These solutions may include outputting decision support information for clinicians to leverage clinician experience and data-driven metrics. The model may output a relative probability that a particular patient may return a particular style of hearing assistance device. By using machine learning techniques (e.g., Bayesian networks), contributions of input variables (e.g., hearing assistance device features or patient information) may be quantified. A clinician may make an informed decision regarding what hearing assistance device or hearing assistance device features to prescribe. Variables that contributed to the final decision of fitting a particular kind of hearing assistance device may be identified and output from the model.

The use of machine learning in the context of patient diagnosis allows for lowering or eliminating variability introduced by lack of patient data (or inability to obtain or use all potentially available patient data), and for enhancing robustness of generalization. The machine learning techniques may include similar execution times with a live clinician diagnosis without machine learning. In an example, there are three operations that are executed when applying machine learning to measured data: collection of data; data preprocessing, and adaptive training of a classifier.

The techniques described herein may use a particular number of 3D shells in a set (e.g., 2, 5, 7, 10, 20, etc.) from a repository of custom-made shells such that one that fits a patient may be predicted. The shells may be mass produced to lower costs and provide an optimized hearing assistance device design.

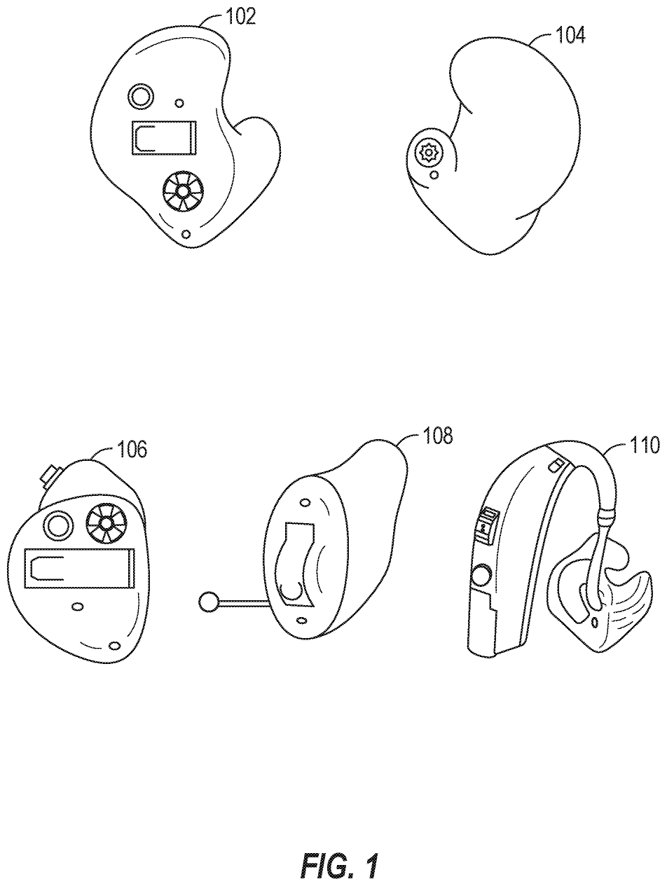

FIG. 1 illustrates examples of hearing assistance devices (e.g., standard or custom) 102-110 according to an example. The example hearing assistance devices include features, which may be combined, rearranged, or removed, and represent examples of different fits and sound delivery. As hearing assistance devices are returned or kept by users over time, a database may be generated including data linked to the return or kept status, such as features of corresponding hearing assistance devices, information about a user of the corresponding hearing assistance devices, reported fit, comfort, or sound quality, or the like.

Stored data about hearing assistance devices may include style (e.g., standard or custom) and features of the hearing assistance device (e.g., directional microphone, digital noise reduction, feedback cancellation, frequency lowering, other physical characteristics, software or firmware version, or other software characteristics). The term style in the context of hearing assistance device may refer to a size, physical appearance, or whether the hearing assistance device is used inside or on the ear. The style of hearing assistance device that is most suitable for an individual may be influenced by many factors, such as the shape of the user's auditory canal, the severity of the user's hearing impairment, the user's dexterity, the user's aesthetic preference, or the like.

Example data used for machine learning includes data from a user to be fitted with a hearing assistance device and stored data about available hearing assistance devices. The data from the user may be collected in a pre-fitting appointments, and may include audiological diagnostic data (e.g., hearing thresholds, word recognition scores, middle ear function testing), hearing assistance device preference data (e.g., hearing assistance device style, dexterity, such as to determine battery size for user), or information about the user (e.g., lifestyle, work environment, age, gender, cognitive ability, personality, financial status, location, hearing assistance device history, or medical history). In an example, audiological diagnostic data may be generated from a standard audiological test, such as conducted by a clinician. Hearing assistance device preference data and user data may be collected via clinical records, for example by the user's written or oral responses to questions. An example of a questionnaire that fulfills such a purpose is shown in Table 1 below.

TABLE 1
Questionnaire to determine patient information

| Query | How assessed | Y/N |
|---|---|---|
| Dexterity problems? | Pill dispenser with batteries. | None/Moderate/Severe |
| | Have patient flip through a | |
| | phone book. | |
| Previous user? | Likes and dislikes regarding | |
| | current amplification | |
| Socially active? | How do you spend your day? | |
| Noisy listening | Are you often in situations | |
| environments? | where there is background | |
| | noise? | |
| Outdoor activities | How much time do you spend | |
| | out doors? | |
| | What outdoor activities do you | |
| | do? | |
| Desire for automatic | Would you prefer to have the | |
| functioning? | hearing aid do everything for | |
| | you or would you like to have | |
| | some control? | |
| Desire for telephone help? | How often do you use the | |
| | telephone? | |
| | Do you have trouble hearing | |
| | over the phone? | |
| | Do you use your hearing aid | |
| | with the phone? | |
| ALD or device compatibility | Do you live alone? | |
| needed? | Do you use any devices to | |
| | help you hear better at home | |
| | or outside home? | |
| | Do you have trouble hearing | |
| | the television? | |
| | Do you have difficulty hearing | |
| | in a large room? | |
| | Do you wish to use | |
| | entertainment devices such as | |
| | MPS, IPOD, etc? | |
| Preference for one or two? | Would you prefer one or two | |
| | hearing aids? | |
| Preference for hearing aid | Do you have an opinion about | |
| style? | the style of the hearing aid? | |
| | Are you concerned about other | |
| | people noticing it? | |
| Wax problems | Do you tend to have a lot of | |
| | wax in your ears? | |
| | Do you often have the wax in | |
| | your ears removed? | |
| Ear drainage | Do you have liquid draining | |
| | from your ears? | |

In an example, a hearing assistance device may include some standard features and optionally some custom features (see Table 2, below for example standard or custom features). For a richer experience, a patient may benefit from adding extra features to the standard configuration of the hearing assistance device. For example, an individual who loves listening to music from a smartphone benefits from a smartphone compatibility feature to allow wirelessly stream music to the hearing assistance device. The standard and custom features shown in Table 2 are examples, and sonic hearing assistance devices may include other standard or custom features.

TABLE 2
Hearing aid feature set

| Category | Features |
|---|---|
| Standard | Battery door |
| | Volume control |
| | Microphone ports |
| | Air vent |
| | Sound outlet |
| | Wax guard |
| | Custom/Standard devices |
| | Ear hook |
| | Plastic tubing |
| | Ear mold |
| | Receiver for Receiver-in-the Canal devices |
| | Removal string |
| | Multiple channels/bands |
| | Multiple memories |
| | Digital noise reduction |
| | Feedback cancellation |
| | Directional microphone |
| Custom | Wireless streaming |
| | Extended bandwidth |
| | Trainable hearing aid technology |
| | Automatic acclimatization |
| | Frequency lowering |
| | Binaural wireless signal processing |
| | Tinnitus sound therapy |
| | Wind noise reduction |
| | Smartphone compatibility |
| | Telecoils |
| | Remote control |

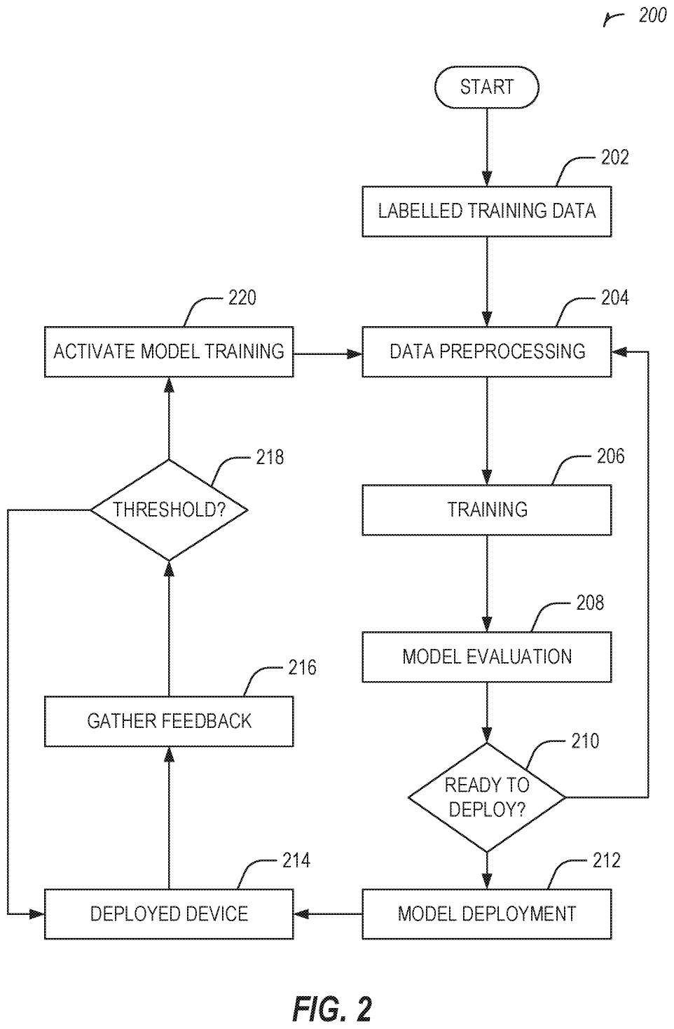

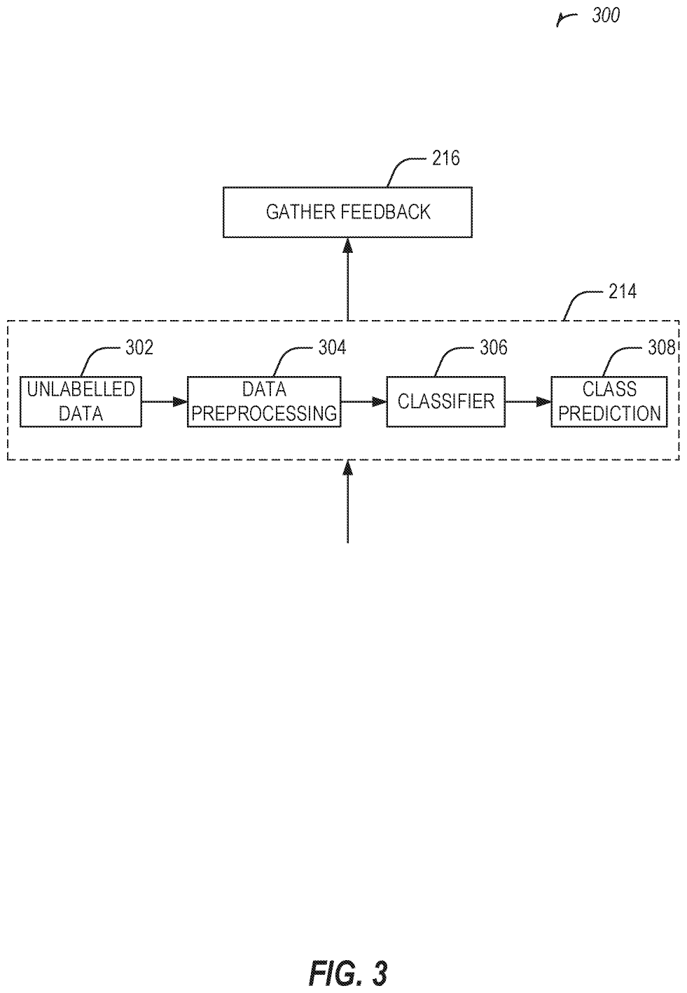

FIGS. 2-3 illustrate machine learning processes 200 and 300 for use in predicting a hearing assistance device shell for a patient according to an example.

To prepare data for machine learning, one approach includes converting raw hearing assistance device style data to a set of useful machine learning features, such as by representing the data as a vector of tokens, for example a vector of 1 s and 0 s. The input to the model may include a matrix of user data, where each row represents a user's record and the columns are derived from concatenation of variables that characterize the user data (e.g., hearing loss, listening demands and lifestyle) and hearing aid features.

Training data may be labeled at operation 202, for example over time to create a database of training data. The machine learning feature sets of training data may be preprocessed at operation 204. Some examples of preprocessing that may be applied here include normalization, applying a transform to the data such that the relevant information is shown first (e.g., kernel transformation or wavelet transform), variable selection (e.g., selecting only features that contribute most to the output of the classifier), or the like. In an example, training data labeled at operation 202 is represented as a vector of tokens of 0 s and 1 s in cases of binary decision (e.g., volume control versus no volume control), while other categorical features may be encoded, for example as a one-hot numeric array (e.g., a 1×N matrix). In another example, any other machine learning feature set may be employed.

Following the pre-processing operation 204, the modified machine learning feature set and corresponding ground truth measurement for at least some of the training data may be fed into a training module at operation 206. Training may be used to estimate an optimal mapping from the feature sets to the corresponding ground truth labels based on some underlying criteria. The resulting mapping (classifier) output from operation 306 may be used to predict a label given the machine learning feature set for unlabeled data 302. The methods by which training may be performed include Logistic Regression, Decision Trees, naive Bayes, Support Vector Machines, Neural Networks, and other Supervised or semi-supervised learning techniques.

The model may be evaluated at operation 208. The classification accuracy may be estimated from an error rate. To avoid overfitting, out-of-sample testing may be used to validate the model. A round of validation may include splitting the training set into k complementary subsets. The classifier is then trained using k−1 of the subsets, leaving one out for testing at operation 208. This may be repeated for all k subsets. The error rate may be obtained as the average of validation results from each subset.

In an example, the model is deployed once optimally trained. In another example, the newly trained model is only deployed if its accuracy exceeds the initial model's accuracy, which may be determined at decision operation 210. In yet another example, when the accuracy is below a certain threshold, the training process 200 continues at operation 212.

As shown in FIG. 2, the model deployment operation 212 prepares the model for deployment by setting up the deployed deep learning device operation 214 with a framework for input and output definition around a machine learning model from the device executing at operation 214. Once deployed, the deployed device executing at operation 214 may execute an input and provide an output. During operation, the input and corresponding output from the execution of the model may be logged at operation 216 until a specified quantity is reached or for a specified period of time at decision operation 218, for example using a threshold. Once this threshold is satisfied, a model retraining may occur at operation 220. Data gathered during this process is used for retraining and the control is sent back to the training module after preprocessing of the new pool of data.

FIG. 3 shows the deployed device operation 214 in more detail. Once a classifier 306 is produced, it may be used to predict the labels of unlabeled data 302. Each portion of unlabeled data 302 is passed to a preprocessing section 304 which accomplishes a similar form of processing as that applied to the training data 202. The classifier 306 is then executed using the modified feature set from preprocessing section 304 as the input to predict the ground truth labels for the data 302. In an example, the output of the model is a class prediction 208 including a number between 0 and 1. A value less than 0.5 may indicate the patient is more likely to return the hearing assistance device and a value greater than 0.5 may indicate a better chance of acceptance.

In some examples, different models may be built based on the demographics of patient data. For example, different models may be built for different regions of the United States, for different markets or countries, for different age groups for different hearing assistance device type (e.g., in ear or on ear), price, or the like.

Referring again to FIG. 2, the training operation 206 may discern clusters of patient data with similar demographics, for example using a centroid-based clustering, distribution-based clustering, density-based clustering, connectivity-based clustering, or any other form of clustering approach. A model may be built at the training operation 206 based on the training data for each patient in a cluster. In an example, the process 200 may assume that individuals with similar demographics will likely gain from similar models.

When patient data is entered into the system, the framework 300 identifies a cluster of that individual and executes the model associated with that cluster, To determine which cluster a new patient data belongs to, within the deployed device operating at 214, a distance function may be used to compute the distance between the mean demographics of each cluster and the demographics of the given patient data. The patient data may be assigned to the cluster with the shortest distance. In an example, when patient data includes demographic data that has a similar distance to the mean demographics of more than one cluster, a mixture of models may be used. For example, different weights may be assigned to each cluster based on the proximity of the demographics of the patient data and respective mean demographics of each cluster or may be based on the probability that the given data belongs to a particular cluster.

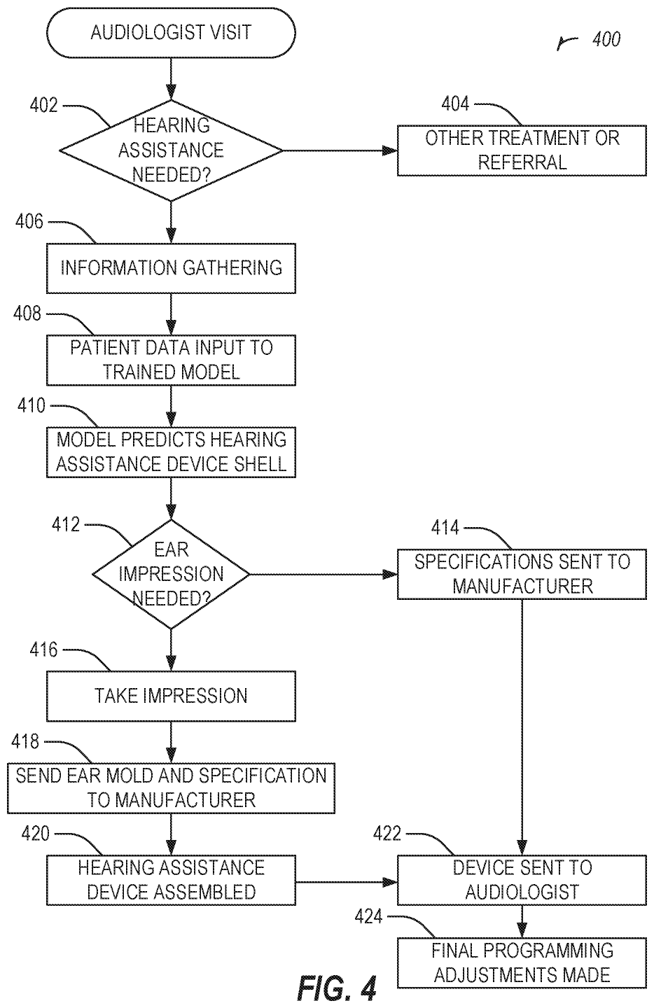

FIG. 4 illustrates a hearing assistance device dispensing process 400 using a prediction model according to an example.

The process 400 illustrates an end-to-end technique for generating a hearing assistance device for a user. Based on the output of a machine learned model, an audiologist may determine whether a custom or a standard hearing assistance device is the best fit for a user. The process 400 includes determining whether the user requires hearing assistance at decision operation 402. When a hearing assistance device is not needed, other treatment or a referral may be provided at operation 404. When a hearing assistance device is needed, information may be gathered (e.g., an audiological test, a questionnaire, etc.) at operation 406. Patient data may be input in a trained model at operation 408, and a predicted hearing assistance device shell may be output via the model at operation 410.

When an ear impression is not needed (e,g., when a standard hearing assistance device is recommended by the model), the specifications may be sent to a manufacturer at operation 414. In a case where a custom hearing assistance device is recommended by the model (e.g., at decision operation 412), the audiologist may make a mold of the user's ear, including the auditory canal, for example using quick hardening silicon 416. The mold provides an impression of the user's ear canal. The requirements of the hearing assistance device or ear canal impression are then sent to the manufacturer for fabrication of a custom hearing device at operation 418.

At a production site, the ear impression is digitized and further processed, such as via a suitable CAD software, into the desired style of hearing assistance device in the specification received. In operation 420, the appropriate electronic components may be customized to address the type of hearing loss diagnosed are placed in the device. Once complete, the hearing assistance device is shipped to the dispensing facility 422 (whether it is a custom or standard hearing assistance device). Another visit to the audiologist may be scheduled by patient for final program adjustments of the hearing aid at operation 424.

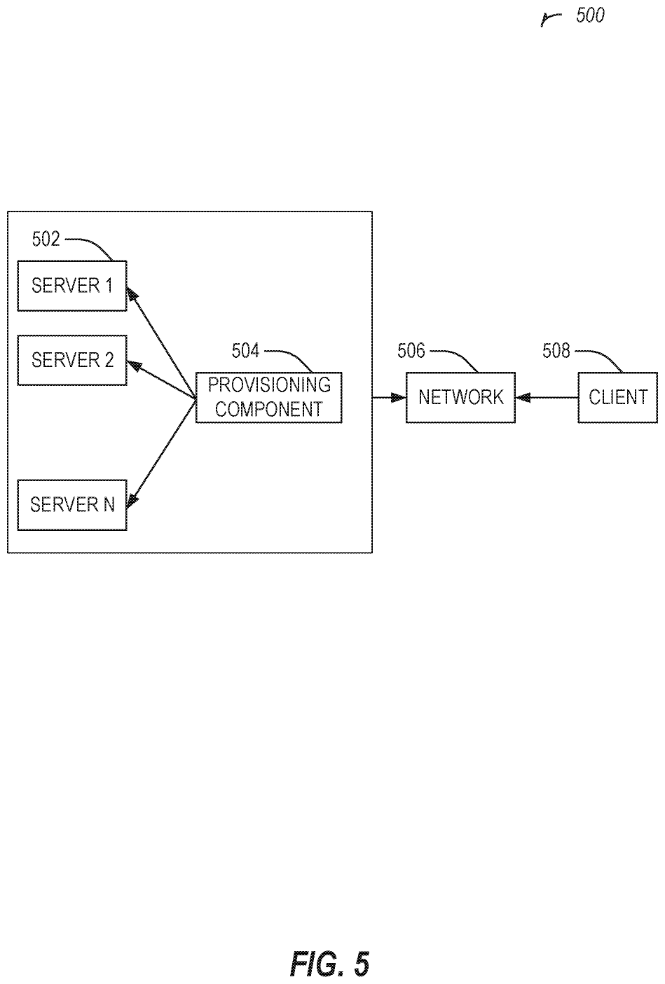

FIG. 5 illustrates a system 500 for predicting hearing assistance device shell for a patient according to an example.

The system 500 may include exemplary hardware that may be used to implement the techniques described herein. System 500 may include one or more servers (e.g., 502) each of, any of, or all of which may work with the provisioning component 504 connected to a network 506, The network 506 may be connected to a client 508. A server node 502 may be any programmable electronic device that is able to receive and send data, such as via network 506. The server node 502 may execute program instructions to run a machine learned model as described herein to output a predicted hearing assistance device.

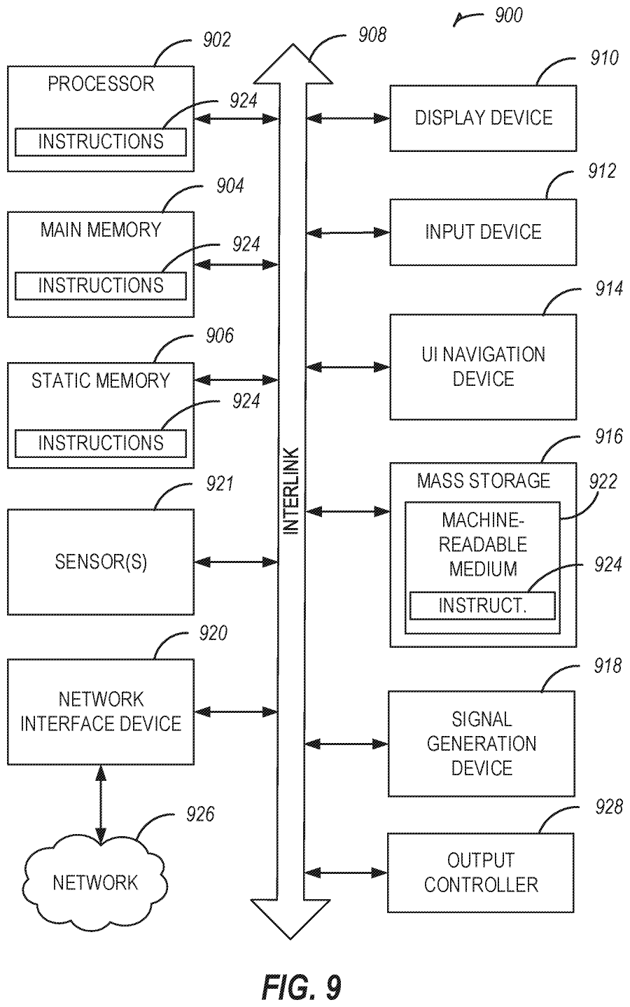

In an example, the one or more servers include one or more databases that store training data used to provide a prediction service via a model to one or more clients 508. In an example, within the provisioning component 504, a machine learning model is built on latest training data. This model is saved in a model store. One or more serving nodes in a serving node cluster may be notified of the availability of an updated model. In one example, the model within one or more sever nodes is automatically updated with a new version when available. In another example, the update may be done in a fixed time interval (e.g., weekly or nightly). Network 506 connects a client 508 to one or more server nodes 502. Network 506 may be any combination of connections or protocols capable of supporting communications between the one or more server nodes 502 and client 508 or the provisioning module 504 and one or more server nodes 502. In an example, client 508 may be a laptop computer, a desktop computer, a smart phone, or any electronic device that is able to communicate with one or more server nodes 502 via the network 506 (e.g., the electronic devices described below with respect to FIG. 9).

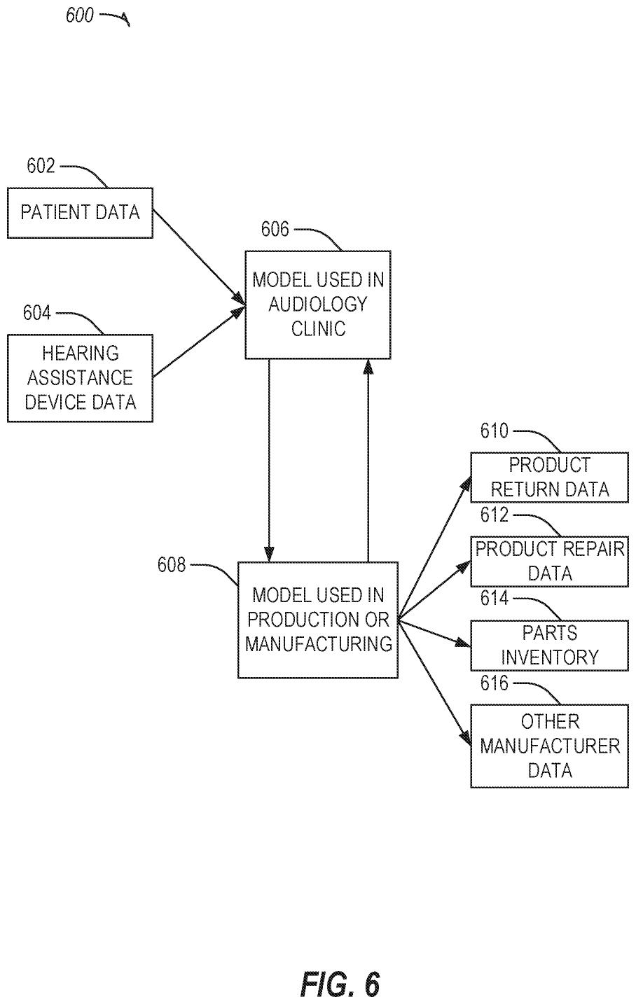

FIG. 6 illustrates prediction models 606 and 608 for clinic and production or manufacturing of hearing assistance devices according to an example.

A prediction model 606 may be used in an audiology clinic or other patient or user interaction setting. A prediction model 608 may be used in production or manufacturing settings. The two prediction models 606 and 608 may, in an example be the same. In this example, the prediction models 606 and 608 may be updated based on data received via either or both settings. In another example, the prediction models 606 and 608 may differ based on different data received via each setting.

Either prediction model 606 or 608 may be built based on existing data regarding product return, repair, ear impression, parts inventory, quality control parts, audiologic information, or the like. For example, when a product order from an audiology clinic is received, this information may be used to modify the prediction model 606 to predict the likelihood of return by using techniques (e.g., earmold scanning and printing techniques) or materials (e.g., silicon materials from suppliers). When the predicted hearing assistance device output by the prediction model 606 and the prediction model 608 differ, a recommendation may be made to the clinician regarding potentially switching to the manufacturer prediction model's 608 recommended hearing assistance device or a recommendation may be made to the manufacturer to switch to the prediction model 606 output.

Various input data may be generated for a particular prediction model 606, such as patient data 602 or hearing assistance device data 604 at the clinic. The clinic itself may be used as input data to the model, for example by weighting the return data or not returned data from past patient data 602 or past hearing assistance device data 604 for that clinic higher than data from other clinics, The manufacturer prediction model 608 may have access to (potentially proprietary) data, such as product return data 610, product repair data 612, parts inventory 614 (e.g., which may be used to eliminate hearing assistance device outputs that are unavailable), or other manufacturer data 616.

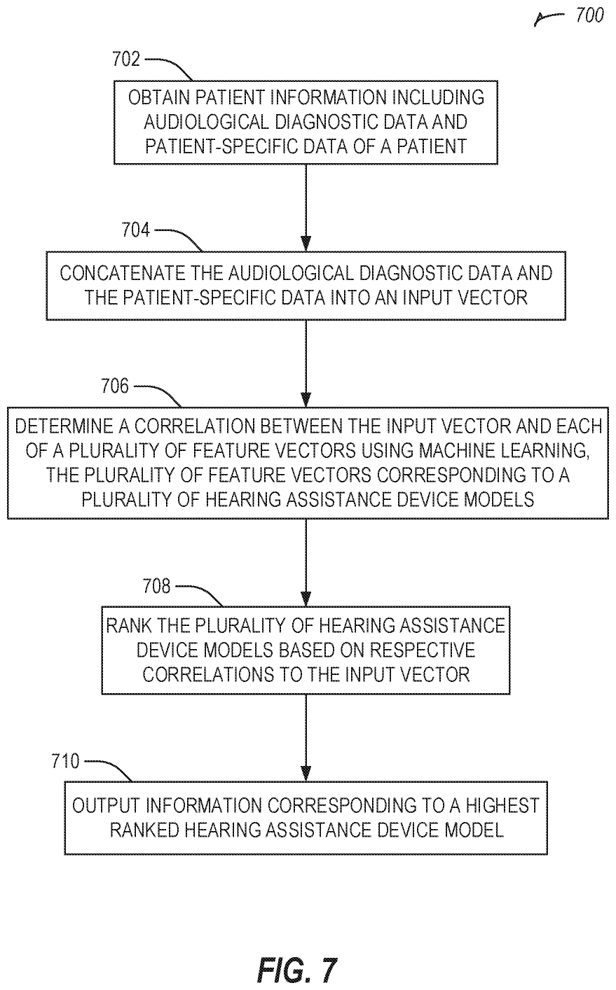

FIG. 7 illustrates a flowchart showing a technique 700 for predicting a hearing assistance device shell for a patient according to an example.

The technique 700 includes an operation 702 to obtain patient information including audiological diagnostic data and patient-specific data of a patient. The patient-specific data may include patient lifestyle, demographic information (e.g., age), hearing aid preference, work environment, cognitive ability, location (e.g., city or state), hearing assistance device history (e.g., previous experience with a hearing assistance device or brand new to hearing assistance devices), medical history data, or the like.

The technique 700 includes an operation 704 to concatenate the audiological diagnostic data and the patient-specific data into an input vector.

The technique 700 includes an operation 706 to determine a correlation between the input vector and each of a plurality of feature vectors using machine learning, the plurality of feature vectors corresponding to a plurality of hearing assistance device models. Operation 706 may include determining the correlation using the input vector and a plurality of feature vectors as inputs for a machine learning trained model. The machine learning trained model may be trained based on a data set including, for example, audiological diagnostic data and patient-specific data corresponding to returned hearing assistance devices or corresponding to hearing assistance devices that were not returned. In this example, the audiological diagnostic data may include at least one of an audiogram, a speech reception threshold, a word recognition score, a middle ear function testing result, or the like.

The technique 700 includes an operation 708 to rank the plurality of hearing assistance device models based on respective correlations to the input vector.

The technique 700 includes an operation 710 to output information corresponding to a highest ranked hearing assistance device model. In an example, outputting the information includes outputting a probability that the highest ranked hearing assistance device model will be returned by the patient, the probability lower than probabilities for other hearing assistance device models in the ranking. In this example, outputting the information includes outputting at least one factor affecting the probability that the highest ranked hearing assistance device model will be returned by the patient. The technique 700 may further include determining at least one feature shared by the highest ranked hearing assistance device model and a next highest ranked hearing assistance device model and outputting an indication of the feature.

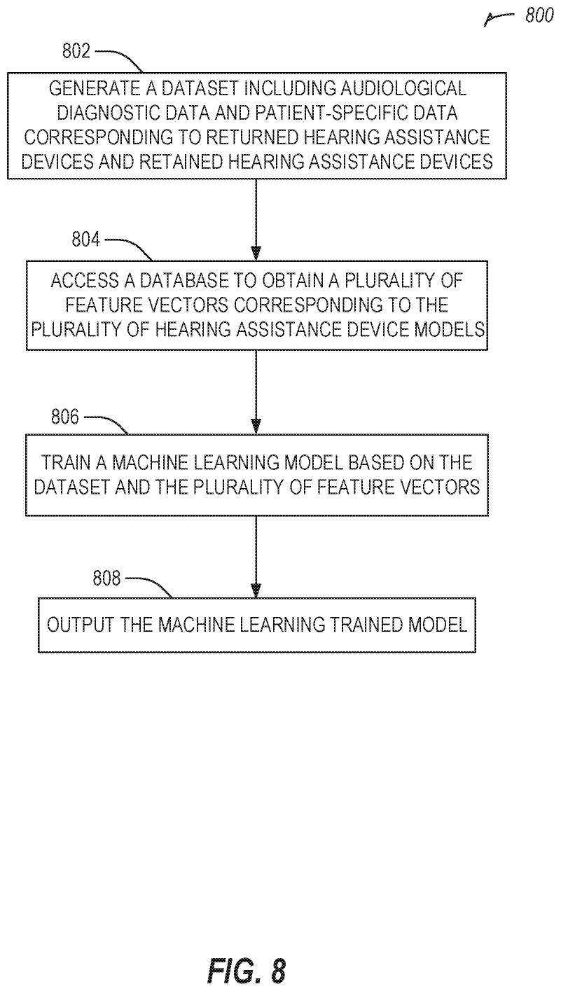

FIG. 8 illustrates a flowchart showing a technique 800 for training a model to predict a hearing assistance device shell for a patient according to an example.

The technique 800 includes an operation 802 to generate a dataset including audiological diagnostic data and patient-specific data corresponding to returned hearing assistance devices and retained hearing assistance devices. In an example, the returned and retained hearing assistance devices are generated from a plurality of hearing assistance device models. The patient-specific data may include patient lifestyle data, demographic data, hearing aid preference data, work environment data, cognitive ability data, location data, financial status data, hearing assistance device history data, medical history data, or the like. The audiological diagnostic data may include an audiogram, a speech reception threshold, a word recognition score, a middle ear function testing result, or the like.

The technique 800 includes an operation 804 to access a database to obtain a plurality of feature vectors corresponding to the plurality of hearing assistance device models. The technique 800 includes an operation 806 to train a machine learning model based on the dataset and the plurality of feature vectors. Operation 806 may include using logistic regression, decision trees, naive Bayes, support vector machines, a neural network (e.g., a recurrent neural network or a convolutional neural network), or the like.

The technique 800 includes an operation 808 to output the machine learning trained model. The machine learning trained model may be configured to rank the plurality of hearing assistance device models based on respective correlations to an input vector including audiological diagnostic data and patient-specific data of a particular patient, for example, The machine learning trained model may be configured to output probabilities that each or any of the plurality of hearing assistance device models will be returned by the particular patient. At least one factor affecting one or more of the probabilities may be output, in an example, such as comfort, fit, style, etc.

FIG. 9 illustrates generally an example of a block diagram of a machine 900 upon which any one or more of the techniques (e.g., methodologies) discussed herein may perform according to an example. In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a hearing assistance device, a personal computer (PC), a tablet PC, a set-top box (SIB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 900 may include a hardware processor 902 (e.g., a central processing unit (CPU), a graphics processing unit (GPU), a hardware processor core, or any combination thereof), a main memory 904 and a static memory 906, some or all of which may communicate with each other via an interlink (e.g., bus) 908. The machine 900 may further include a display unit 910, an alphanumeric input device 912 (e.g., a keyboard), and a user interface (UI) navigation device 914 (e.g., a mouse). In an example, the display unit 910, alphanumeric input device 912 and UI navigation device 914 may be a touch screen display. The machine 900 may additionally include a storage device (e.g., drive unit) 916, a signal generation device 918 (e.g., a speaker), a network interface device 920, and one or more sensors 921, such as a global positioning system (GPS) sensor, compass, accelerometer, or other sensor. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 916 may include a machine readable medium 922 that is non-transitory on which is stored one or more sets of data structures or instructions 924 (e.g., software) embodying or utilized by any one or more of the techniques or functions described herein. The instructions 924 may also reside, completely or at least partially, within the main memory 904, within static memory 906, or within the hardware processor 902 during execution thereof by the machine 900. In an example, one or any combination of the hardware processor 902, the main memory 904, the static memory 906, or the storage device 916 may constitute machine readable media.

While the machine readable medium 922 is illustrated as a single medium, the term “machine readable medium” may include a single medium or multiple media (e.g., a centralized or distributed database, and/or associated caches and servers) configured to store the one or more instructions 924.

The term “machine readable medium” may include any medium that is capable of storing, encoding, or carrying instructions for execution by the machine 900 and that cause the machine 900 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may further be transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 900, and includes digital or analog communications signals or other intangible medium to facilitate communication of such software.

Hearing assistance devices typically include at least one enclosure or housing, a microphone, hearing assistance device electronics including processing electronics, and a speaker or “receiver.” Hearing assistance devices may include a power source, such as a battery. In various embodiments, the battery may be rechargeable. In various embodiments multiple energy sources may be employed. It is understood that in various embodiments the microphone is optional. It is understood that in various embodiments the receiver is optional. It is understood that variations in communications protocols, antenna configurations, and combinations of components may be employed without departing from the scope of the present subject matter. Antenna configurations may vary and may be included within an enclosure for the electronics or be external to an enclosure for the electronics. Thus, the examples set forth herein are intended to be demonstrative and not a limiting or exhaustive depiction of variations.

It is understood that digital hearing assistance devices include a processor. In digital hearing assistance devices with a processor, programmable gains may be employed to adjust the hearing assistance device output to a wearer's particular hearing impairment. The processor may be a digital signal processor (DSP), microprocessor, microcontroller, other digital logic, or combinations thereof. The processing may be done by a single processor, or may be distributed over different devices. The processing of signals referenced in this application may be performed using the processor or over different devices. Processing may be done in the digital domain, the analog domain, or combinations thereof. Processing may be done using subband processing techniques. Processing may be done using frequency domain or time domain approaches. Some processing may involve both frequency and time domain aspects. For brevity, in some examples drawings may omit certain blocks that perform frequency synthesis, frequency analysis, analog-to-digital conversion, digital-to-analog conversion, amplification, buffering, and certain types of filtering and processing. In various embodiments the processor is adapted to perform instructions stored in one or more memories, which may or may not be explicitly shown. Various types of memory may be used, including volatile and nonvolatile forms of memory. In various embodiments, the processor or other processing devices execute instructions to perform a number of signal processing tasks. Such embodiments may include analog components in communication with the processor to perform signal processing tasks, such as sound reception by a microphone, or playing of sound using a receiver (i.e., in applications where such transducers are used). In various embodiments, different realizations of the block diagrams, circuits, and processes set forth herein may be created by one of skill in the art without departing from the scope of the present subject matter.

Various embodiments of the present subject matter support wireless communications with a hearing assistance device. In various embodiments the wireless communications may include standard or nonstandard communications. Some examples of standard wireless communications include, but not limited to, Bluetooth™, low energy Bluetooth, IEEE 802.11(wireless LANs), 802.15 (WPANs), and 802.16 (WiMAX). Cellular communications may include, but not limited to, CDMA, GSM, ZigBee, and ultra-wideband (UWB) technologies. In various embodiments, the communications are radio frequency communications. In various embodiments the communications are optical communications, such as infrared communications. In various embodiments, the communications are inductive communications. In various embodiments, the communications are ultrasound communications. Although embodiments of the present system may be demonstrated as radio communication systems, it is possible that other forms of wireless communications may be used. It is understood that past and present standards may be used. It is also contemplated that future versions of these standards and new future standards may be employed without departing from the scope of the present subject matter.

The wireless communications support a connection from other devices. Such connections include, but are not limited to, one or more mono or stereo connections or digital connections having link protocols including, but not limited to 802.3 (Ethernet), 802.4, 802.5, USB, ATM, Fibre-channel, Firewire or 1394, InfiniBand, or a native streaming interface, In various embodiments, such connections include all past and present link protocols. It is also contemplated that future versions of these protocols and new protocols may be employed without departing from the scope of the present subject matter.

In various embodiments, the present subject matter is used in hearing assistance devices that are configured to communicate with mobile phones. In such embodiments, the hearing assistance device may be operable to perform one or more of the following: answer incoming calls, hang up on calls, and/or provide two way telephone communications. In various embodiments, the present subject matter is used in hearing assistance devices configured to communicate with packet-based devices. In various embodiments, the present subject matter includes hearing assistance devices configured to communicate with streaming audio devices. In various embodiments, the present subject matter includes hearing assistance devices configured to communicate with Wi-Fi devices. In various embodiments, the present subject matter includes hearing assistance devices capable of being controlled by remote control devices.

It is further understood that different hearing assistance devices may embody the present subject matter without departing from the scope of the present disclosure. The devices depicted in the figures are intended to demonstrate the subject matter, but not necessarily in a limited, exhaustive, or exclusive sense. It is also understood that the present subject matter may be used with a device designed for use in the right ear or the left ear or both ears of the wearer.

The present subject matter may be employed in hearing assistance devices, such as headsets, headphones, and similar hearing devices.

The present subject matter is demonstrated for hearing assistance devices, including hearing assistance devices, including but not limited to, behind-the-ear (BTE), in-the-ear (ITE), in-the-canal (ITC), receiver-in-canal (RIC), or completely-in-the-canal (CIC) type hearing assistance devices. It is understood that behind-the-ear type hearing assistance devices may include devices that reside substantially behind the ear or over the ear. Such devices may include hearing assistance devices with receivers associated with the electronics portion of the behind-the-ear device, or hearing assistance devices of the type having receivers in the ear canal of the user, including but not limited to receiver-in-canal (RIC) or receiver-in-the-ear (RITE) designs. The present subject matter may also be used in hearing assistance devices generally, such as cochlear implant type hearing devices and such as deep insertion devices having a transducer, such as a receiver or microphone, whether custom fitted, standard fitted, open fitted and/or occlusive fitted. It is understood that other hearing assistance devices not expressly stated herein may be used in conjunction with the present subject matter.

Each of the following non-limiting examples may stand on its own, or may be combined in various permutations or combinations with one or more of the other examples.

Example 1 is a method comprising: obtaining patient information including audiological diagnostic data and patient-specific data of a patient; concatenating the audiological diagnostic data and the patient-specific data into an input vector; determining, using the input vector and a plurality of feature vectors as inputs for a machine learning trained model, a correlation between the input vector and each of the plurality of feature vectors, the plurality of feature vectors corresponding to a plurality of hearing assistance device models; ranking the plurality of hearing assistance device models based on respective correlations to the input vector; and outputting information corresponding to a highest ranked hearing assistance device model.

In Example 2, the subject matter of Example 1 includes, wherein the patient-specific data includes at least one of patient lifestyle, demographic, hearing aid preference, work environment, cognitive ability, location, financial status, hearing assistance device history, or medical history data.

In Example 3, the subject matter of Examples 1-2 includes, wherein outputting the information includes outputting a probability that the highest ranked hearing assistance device model will be returned by the patient, the probability lower than probabilities for other hearing assistance device models in the ranking.

In Example 4, the subject matter of Example 3 includes, wherein outputting the information includes outputting at least one factor affecting the probability that the highest ranked hearing assistance device model will be returned by the patient.

In Example 5, the subject matter of Examples 1-4 includes, wherein the machine learning trained model is trained based on a data set including audiological diagnostic data and patient-specific data corresponding to returned hearing assistance devices and audiological diagnostic data and patient-specific data corresponding to hearing assistance devices that were not returned.

In Example 6, the subject matter of Examples 1-5 includes, wherein the audiological diagnostic data includes at least one of an audiogram, a speech reception threshold, a word recognition score, or a middle ear function testing result.

In Example 7, the subject matter of Examples 1-6 includes, determining at least one feature shared by the highest ranked hearing assistance device model and a next highest ranked hearing assistance device model and outputting an indication of the feature.

Example 8 is a system comprising: one or more processors coupled to a memory device, the memory device containing instructions which, when executed by the one or more processors, cause the system to: obtain patient information including audiological diagnostic data and patient-specific data of a patient; concatenate the audiological diagnostic data and the patient-specific data into an input vector; determine, using the input vector and a plurality of feature vectors as inputs for a machine learning trained model, a correlation between the input vector and each of the plurality of feature vectors, the plurality of feature vectors corresponding to a plurality of hearing assistance device models; rank the plurality of hearing assistance device models based on respective correlations to the input vector; and output information corresponding to a highest ranked hearing assistance device model.

In Example 9, the subject matter of Example 8 includes, wherein the patient-specific data includes at least one of patient lifestyle, demographic, hearing aid preference, work environment, cognitive ability, location, financial status, hearing assistance device history, or medical history data.

In Example 10, the subject matter of Examples 8-9 includes, wherein to output the information, the instructions further cause the system to output a probability that the highest ranked hearing assistance device model will be returned by the patient, the probability lower than probabilities for other hearing assistance device models in the ranking.

In Example 11, the subject matter of Example 10 includes, wherein to output the information, the instructions further cause the system to output at least one factor affecting the probability that the highest ranked hearing assistance device model will be returned by the patient.

In Example 12, the subject matter of Examples 8-11 includes, wherein the machine learning trained model is trained based on a data set including audiological diagnostic data and patient-specific data corresponding to returned hearing assistance devices and audiological diagnostic data and patient-specific data corresponding to hearing assistance devices that were not returned.

In Example 13, the subject matter of Examples 8-12 includes, wherein the audiological diagnostic data includes at least one of an audiogram, a speech reception threshold, a word recognition score, or a middle ear function testing result.

In Example 14, the subject matter of Examples 8-13 includes, wherein the instructions further cause the system to determine at least one feature shared by the highest ranked hearing assistance device model and a next highest ranked hearing assistance device model and outputting an indication of the feature.

Example 15 is a method comprising; generating a dataset including audiological diagnostic data and patient-specific data corresponding to returned hearing assistance devices, and audiological diagnostic data and patient-specific data corresponding to retained hearing assistance devices, the returned and retained hearing assistance devices generated from a plurality of hearing assistance device models; accessing a database to obtain a plurality of feature vectors corresponding to the plurality of hearing assistance device models; training a machine learning model based on the dataset and the plurality of feature vectors; and outputting the machine learning trained model, the machine learning trained model configured to rank the plurality of hearing assistance device models based on respective correlations to an input vector including audiological diagnostic data and patient-specific data of a particular patient.

In Example 16, the subject matter of Example 15 includes, wherein the patient-specific data includes at least one of patient lifestyle, demographic, hearing aid preference, work environment, cognitive ability, location, financial status, hearing assistance device history, or medical history data.

In Example 17, the subject matter of Examples 15-16 includes, wherein the machine learning trained model is configured to output probabilities that each of the plurality of hearing assistance device models will be returned by the particular patient.

In Example 18, the subject matter of Example 17 includes, wherein the machine learning trained model is configured to output at least one factor affecting the probabilities.

In Example 19, the subject matter of Examples 15-18 includes, wherein the audiological diagnostic data includes at least one of an audiogram, a speech reception threshold, a word recognition score, or a middle ear function testing result.

In Example 20, the subject matter of Examples 15-19 includes, wherein training the machine learning trained model includes using logistic regression, decision trees, naive Bayes, support vector machines, or a neural network.

Example 21 is a system comprising: one or more processors coupled to a memory device, the memory device containing instructions which, when executed by the one or more processors, cause the system to: generate a dataset including audiological diagnostic data and patient-specific data corresponding to returned hearing assistance devices, and audiological diagnostic data and patient-specific data corresponding to retained hearing assistance devices, the returned and retained hearing assistance devices generated from a plurality of hearing assistance device models; access a database to obtain a plurality of feature vectors corresponding to the plurality of hearing assistance device models; train a machine learning model based on the dataset and the plurality of feature vectors; and output the machine learning trained model, the machine learning trained model configured to rank the plurality of hearing assistance device models based on respective correlations to an input vector including audiological diagnostic data and patient-specific data of a particular patient.

In Example 22, the subject matter of Example 21 includes, wherein the patient-specific data includes at least one of patient lifestyle, demographic, hearing aid preference, work environment, cognitive ability, location, financial status, hearing assistance device history, or medical history data.

In Example 23, the subject matter of Examples 21-22 includes, wherein the machine learning trained model is configured to output probabilities that each of the plurality of hearing assistance device models will be returned by the particular patient.

In Example 24, the subject matter of Example 23 includes, wherein the machine learning trained model is configured to output at least one factor affecting the probabilities.

In Example 25, the subject matter of Examples 21-24 includes, wherein the audiological diagnostic data includes at least one of an audiogram, a speech reception threshold, a word recognition score, or a middle ear function testing result.

In Example 26, the subject matter of Examples 21-25 includes, wherein to train the machine learning trained model, the instructions further cause the system to use logistic regression, decision trees, naive Bayes, support vector machines, or a neural network.

Example 27 is at least one machine-readable medium including instructions that, when executed by processing circuitry, cause the processing circuitry to perform operations to implement of any of Examples 1-26.

Example 28 is an apparatus comprising means to implement of any of Examples 1-26.

Example 29 is a system to implement of any of Examples 1-26.

Example 30 is a method to implement of any of Examples 1-26.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

1. A method comprising:
obtaining patient information including audiological diagnostic data and patient-specific data of a patient;
concatenating the audiological diagnostic data and the patient-specific data into an input vector;
determining, using the input vector and a plurality of feature vectors as inputs for a machine learning trained model, a correlation between the input vector and each of the plurality of feature vectors, the plurality of feature vectors corresponding to a plurality of hearing assistance device models;
ranking the plurality of hearing assistance device models based on respective correlations to the input vector; and
outputting information corresponding to a highest ranked hearing assistance device model.

2. The method of claim 1, wherein the patient-specific data includes at least one of patient lifestyle, demographic, hearing aid preference, work environment, cognitive ability, location, financial status, hearing assistance device history, or medical history data.

3. The method of claim 1, wherein outputting the information includes outputting a probability that the highest ranked hearing assistance device model will be returned by the patient, the probability lower than probabilities for other hearing assistance device models in the ranking.

4. The method of claim 3, wherein outputting the information includes outputting at least one factor affecting the probability that the highest ranked hearing assistance device model will be returned by the patient.

5. The method of claim 1, wherein the machine learning trained model is trained based on a data set including audiological diagnostic data and patient-specific data corresponding to returned hearing assistance devices and audiological diagnostic data and patient-specific data corresponding to hearing assistance devices that were not returned.

6. The method of claim 1, wherein the audiological diagnostic data includes at least one of an audiogram, a speech reception threshold, a word recognition score, or a middle ear function testing result.

7. The method of claim 1, further comprising determining at least one feature shared by the highest ranked hearing assistance device model and a next highest ranked hearing assistance device model and outputting an indication of the feature.

8. A system comprising:
one or more processors coupled to a memory device, the memory device containing instructions which, when executed by the one or more processors, cause the system to:
obtain patient information including audiological diagnostic data and patient-specific data of a patient;
concatenate the audiological diagnostic data and the patient-specific data into an input vector;
determine, using the input vector and a plurality of feature vectors as inputs for a machine learning trained model, a correlation between the input vector and each of the plurality of feature vectors, the plurality of feature vectors corresponding to a plurality of hearing assistance device models;
rank the plurality of hearing assistance device models based on respective correlations to the input vector; and
output information corresponding to a highest ranked hearing assistance device model.

9. The system of claim 8, wherein the patient-specific data includes at least one of patient lifestyle, demographic, hearing aid preference, work environment, cognitive ability, location, financial status, hearing assistance device history, or medical history data.

10. The system of claim 8, wherein to output the information, the instructions further cause the system to output a probability that the highest ranked hearing assistance device model will be returned by the patient, the probability lower than probabilities for other hearing assistance device models in the ranking.

11. The system of claim 10, wherein to output the information, the instructions further cause the system to output at least one factor affecting the probability that the highest ranked hearing assistance device model will be returned by the patient.

12. The system of claim 8, wherein the machine learning trained model is trained based on a data set including audiological diagnostic data and patient-specific data corresponding to returned hearing assistance devices and audiological diagnostic data and patient-specific data corresponding to hearing assistance devices that were not returned.

13. The system of claim 8, wherein the audiological diagnostic data includes at least one of an audiogram, a speech reception threshold, a word recognition score, or a middle ear function testing result.

14. The system of claim 8, wherein the instructions further cause the system to determine at least one feature shared by the highest ranked hearing assistance device model and a next highest ranked hearing assistance device model and outputting an indication of the feature.

15. A method comprising:
generating a dataset including audiological diagnostic data and patient-specific data corresponding to returned hearing assistance devices, and audiological diagnostic data and patient-specific data corresponding to retained hearing assistance devices, the returned and retained hearing assistance devices generated from a plurality of hearing assistance device models;
accessing a database to obtain a plurality of feature vectors corresponding to the plurality of hearing assistance device models;
training a machine learning model based on the dataset and the plurality of feature vectors; and
outputting the machine learning trained model, the machine learning trained model configured to rank the plurality of hearing assistance device models based on respective correlations to an input vector including audiological diagnostic data and patient-specific data of a particular patient.

16. The method of claim 15, wherein the patient-specific data includes at least one of patient lifestyle, demographic, hearing aid preference, work environment, cognitive ability, location, financial status, hearing assistance device history, or medical history data.

17. The method of claim 15, wherein the machine learning trained model is configured to output probabilities that each of the plurality of hearing assistance device models will be returned by the particular patient.

18. The method of claim 17, wherein the machine learning trained model is configured to output at least one factor affecting the probabilities.

19. The method of claim 15, wherein the audiological diagnostic data includes at least one of an audiogram, a speech reception threshold, a word recognition score, or a middle ear function testing result.

20. The method of claim 15, wherein training the machine learning trained model includes using logistic regression, decision trees, naive Bayes, support vector machines, or a neural network.
